(19) United States
(12) Patent Application Publication
(10) Pub. No.: US 2004/0187564 A1
(43) Pub. Date: Sep. 30, 2004

(54) Dateable inks

(76) Inventor: Jon C. Hagerty, Chelsea, VT (US)

Correspondence Address: JON C. HAGERTY, BOX 183, CHELSEA, VT, 05038, US

(21) Appl. No.: 10/767,142

(22) Filed: Jan. 29, 2004

Related U.S. Application Data
(60) Provisional application No. 60/458,601, filed on Mar. 28, 2003.

(51) Int. Cl. G01N 33/32

(52) U.S. Cl. 73/61.41; 252/408.1; 252/965

(57) ABSTRACT

Additives such as chemical elements, microstructures, time degradable molecules, micro oscillators (clocks) etc. are introduced into inks at production. The additives can be analyzed in samples thus giving source of and date of production. By varying additives over short time spans estimates of date of use may be obtained.

### CROSS REFERENCE TO RELATED CASES

This application claims benefit of U.S. provisional patent serial No. 60/458601, filed on Mar. 28, 2003.

### BACKGROUND

1. Field of Invention

This invention relates to methods for dating inks.

2. Description of Prior Art

No prior art has been found.

### SUMMARY

Inks are produced with additives. Samples of inks can be analyzed to give source and date of production as well as estimated date of use.

### DESCRIPTION

Additives such as chemical elements, microstructures, time degradable molecules, micro oscillators (clocks) etc. are introduced to inks at production. The additives date and show the source of production. By varying additives over short time periods estimates of date of use may be obtained.

### Conclusions, Ramifications and Scope

The invention described herein relates to dateable inks. Additives are introduced into inks at production. Analyses can then determine source and date of production as well as estimated date of use.

The embodiments described are not intended to limit the scope and application of this patent.

What is claimed is:

1. Methods for dating inks wherein additives are introduced to inks at production.

2. Qualitative and/or quantities systems to analyze ink samples for additives thus dating, showing source of production and estimates of time of use.

3. A method of varying additives over short periods to approximate date of use.
